(19) United States
(12) Patent Application Publication
(10) Pub. No.: US 2013/0023750 A1
(43) Pub. Date: Jan. 24, 2013

(54) ECG ELECTRODE SYSTEM

(71) Applicant: Mark J. Callahan, Medway, MA (US)

(72) Inventor: Mark J. Callahan, Medway, MA (US)

(73) Assignee: TYCO HEALTHCARE GROUP LP, Mansfield, MA (US)

(21) Appl. No.: 13/553,135

(22) Filed: Jul. 19, 2012

Related U.S. Application Data
(60) Provisional application No. 61/510,527, filed on Jul. 22, 2011.

(51) Int. Cl. A61B 5/0408

(52) U.S. Cl. 600/386; 600/509

(57) ABSTRACT

An ECG electrode lead system suitable for use during imaging procedures such as, without limitation, CT scans or MRI and methods of use. The a radiolucent ECG lead set cable includes at least one radiolucent conductor, at least one radiolucent electrode connector operatively coupled to a distal end of the ECG lead set cable, an ECG intermediate lead set connector disposed at a proximal end of the ECG lead set cable, and an ECG lead extension assembly.

### CROSS-REFERENCE TO RELATED APPLICATION

This application claims the benefit of and priority to U.S. Provisional Application Ser. No. 61/510,527, filed on Jul. 22, 2011, the entire contents of which is hereby incorporated by reference herein for all purposes.

### BACKGROUND

1. Technical Field

The present disclosure relates to biomedical electrodes, and in particular, to a radiolucent biomedical electrode connector and radiolucent lead wires for performing biomedical monitoring of a patent during imaging procedures.

2. Background of Related Art

Electrocardiograph (ECG) monitors are widely used to obtain medical (i.e. biopotential) signals containing information indicative of the electrical activity associated with the heart and pulmonary system. To obtain medical signals, ECG electrodes are applied to the skin of a patient in various locations. The electrodes, after being positioned on the patient, connect to an ECG monitor by a set of ECG lead wires. The distal end of the ECG lead wire, or portion closest to the patient, may include a connector which is adapted to operably connect to the electrode to receive medical signals from the body. The proximal end of the ECG lead set is operably coupled to the ECG monitor either directly or indirectly through an adapter, and supplies the medical signals received from the body to the ECG monitor.

A typical ECG electrode assembly may include an electrically conductive layer and a backing layer, the assembly having a patient contact side and a connector side. The contact side of the electrode pad may include biocompatible conductive gel or adhesive for affixing the electrode to a patient's body for facilitating an appropriate electrical connection between a patient's body and the electrode assembly. The connector side of the pad may incorporate a metallic press stud having a bulbous profile for coupling the electrode pad to the ECG lead wire. In use, the clinician removes a protective covering from the electrode side to expose the gel or adhesive, affixes the electrode pad to the patient's body, and attaches the appropriate ECG lead wire connector to the press stud by pressing or “snapping” the lead wire connector onto the bulbous press stud to achieve mechanical and electrical coupling of the electrode and lead wire. Alternatively, ECG connectors that engage via manipulation of a lever or other mechanical locking device may be employed. After use, a clinician then removes the ECG lead wire connector from the pad by pulling or “unsnapping” the connector from the pad or by releasing the lever or other locking mechanism.

Placement of the electrodes on a patient has been established by medical protocols. A common protocol requires the placement of the electrodes in a 5-lead configuration: one electrode adjacent each clavicle bone on the upper chest and a third electrode adjacent the patient's lower left abdomen, a fourth electrode adjacent the sternum, and a fifth electrode on the patient's lower right abdomen.

During certain procedures it may be necessary to monitor biological (e.g., ECG) parameters of a patient that is undergoing imaging, such as CT-scan or MRI. Use of conventional ECG connectors and lead wire sets typically associated therewith may have drawbacks in these applications, since they tend to interfere with the imaging systems. In one example, certain components of the ECG connectors and/or lead wires may be detected by the imaging apparatus and consequently may obfuscate the visual images upon which clinicians and surgeons rely. In another example, ferrous and/or magnetic components commonly found in ECG connectors, such as in springs and clips, may be potentially hazardous when used within the intense magnetic field of an MRI scanner.

### SUMMARY

In an embodiment in accordance with the present disclosure, there is provided an ECG lead system that, in accordance with embodiments of the present disclosure, comprises a radiolucent ECG lead set assembly and an ECG lead extension assembly. The ECG lead set assembly comprises a radiolucent ECG lead set cable having at least one radiolucent conductor. At least one radiolucent electrode connector is operatively coupled to a distal end of the ECG lead set cable, and an ECG intermediate lead set connector is disposed at a proximal end of the ECG lead set cable. The ECG lead extension assembly comprises an ECG lead extension cable having at least one conductor. An ECG lead set extension connector is disposed at a distal end of the ECG lead extension cable, and a device connector is disposed at a proximal end of the ECG lead extension cable. The ECG intermediate lead set connector is configured to operatively couple to the ECG lead set extension connector. The device connector is configured to operatively couple to an ECG monitor.

A method of performing an ECG on a patient undergoing an imaging procedure is provided. In embodiments according to the present disclosure, the method comprises providing one or more radiolucent ECG connectors as described herein, providing a radiolucent ECG lead system as described herein, attaching one or more electrode pads to the body of a patient, operatively coupling the one or more radiolucent ECG connectors to a corresponding one of th one or more electrode pads, operatively coupling the device connector to an ECG monitor, and imaging the patient in an imaging apparatus selected from the group consisting of an MRI scanner, a CT scanner, and a PET scanner. The method in may include coupling the ECG intermediate lead set connector to the ECG lead set extension connector. Additionally or alternatively, the method may include providing an adapter configured to enable operable coupling of the device connector to an ECG monitor, coupling the device connector to the adapter, and coupling the adapter to the ECG monitor.

### BRIEF DESCRIPTION OF THE DRAWINGS

Various embodiments of the present disclosure are described hereinbelow with references to the drawings, wherein:

FIG. 1A is a view of an embodiment of a radiolucent ECG electrode connector in an engaged configuration in accordance with the present disclosure;

FIG. 1B is a view of the FIG. 1A embodiment in a disengaged configuration in accordance with the present disclosure;

FIG. 1C is a detail view of a press stud opening of the FIG. 1A embodiment of a radiolucent ECG electrode connector in accordance with the present disclosure;

FIG. 2A is a view of another embodiment of a radiolucent ECG electrode connector in an engaged configuration in accordance with the present disclosure;

FIG. 2B is a view of the FIG. 2A embodiment in a disengaged configuration in accordance with the present disclosure;

FIG. 3 is a view of another embodiment of a radiolucent ECG electrode connector in accordance with the present disclosure;

FIG. 4A is a view of an embodiment of a dual-section ECG electrode wiring harness in accordance with the present disclosure;

FIG. 4B is a cross-sectional view of a portion of the dual-section ECG electrode wiring harness of FIG. 4A; and

FIG. 5 is a view of a dual-section ECG electrode wiring harness in accordance with the present disclosure during use.

### DETAILED DESCRIPTION OF EMBODIMENTS

Particular embodiments of the present disclosure are described hereinbelow with reference to the accompanying drawings; however, the disclosed embodiments are merely examples of the disclosure, which may be embodied in various forms. Well-known functions or constructions and repetitive matter are not described in detail to avoid obscuring the present disclosure in unnecessary or redundant detail. Therefore, specific structural and functional details disclosed herein are not to be interpreted as limiting, but merely as a basis for the claims and as a representative basis for teaching one skilled in the art to variously employ the present disclosure in virtually any appropriately detailed structure. In this description, as well as in the drawings, like-referenced numbers represent elements which may perform the same, similar, or equivalent functions.

In the drawings and in the descriptions that follow, the term “proximal,” as is traditional, shall refer to the end of the instrument that is closer to a user, while the term “distal” shall refer to the end that is farther from a user. In addition, as used herein, terms referencing orientation, e.g., “top”, “bottom”, “up”, “down”, “left”, “right”, “clockwise”, “counterclockwise”, and the like, are used for illustrative purposes with reference to the figures and features shown therein. Embodiments in accordance with the present disclosure may be practiced in any orientation without limitation.

The present invention is directed to an electrode system suitable for use during patient imaging, such as during a CT-scan or MRI. Commonly available electrode connectors have components which may be detected on the image and/or may become dangerous when exposed to a particular field, such as a magnetic field.

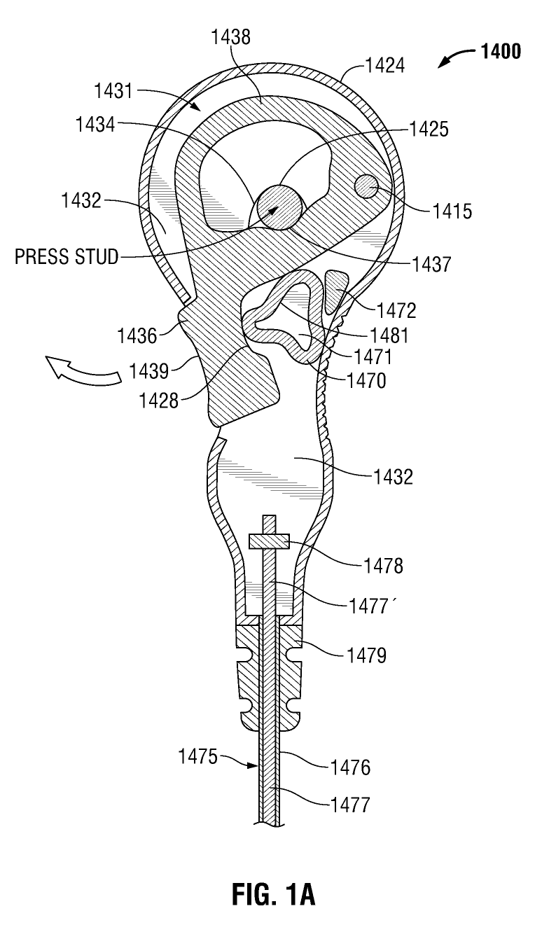

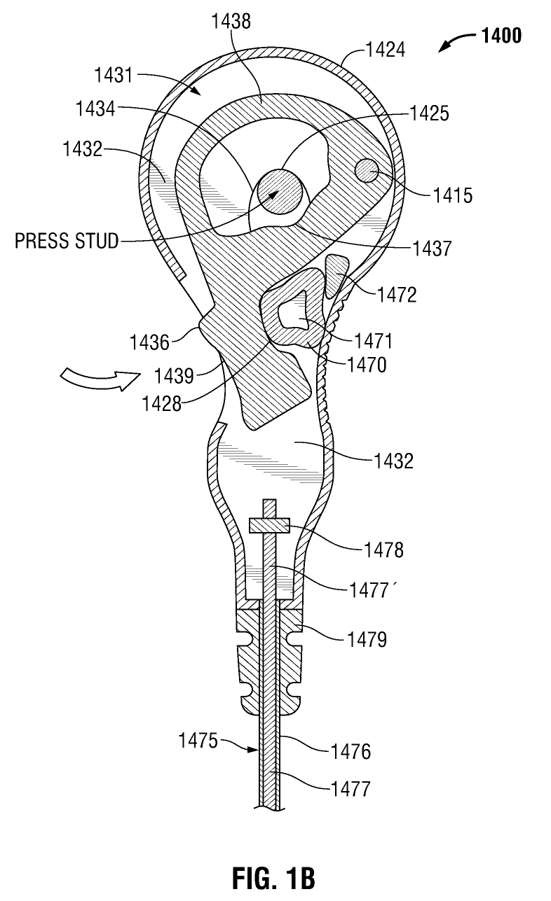

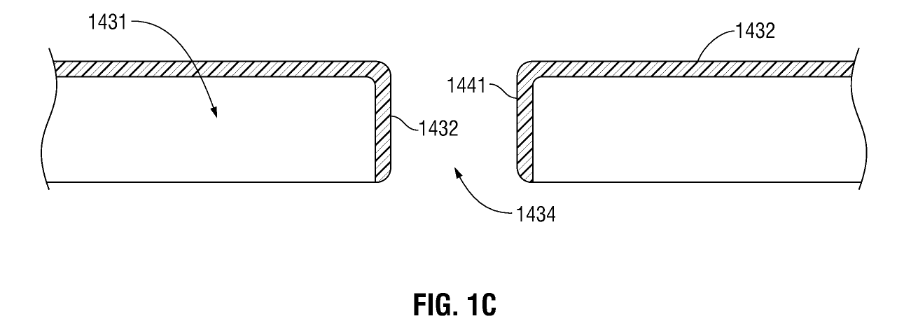

Accordingly, one aspect of the present invention provides an electrode connector which may be used during patient imaging. One embodiment of an ECG electrode connector of the present invention is shown in FIGS. 1A, 1B, and 1C. In view thereof, and so as not to obscure the present disclosure with redundant information, only those features distinct to ECG electrode connector 1400 will be described hereinafter.

ECG electrode connector 1400 is configured to facilitate the monitoring of ECG and other biological parameters while the subject patient is undergoing an imaging procedure, such as without limitation, MRI, CT, PET, and the like. Connector 1400 includes a housing 1424 having an interior recessed surface 1431 that includes an opening 1434 defined therein that opens to a patient-facing surface of the housing. Opening 1434 is dimensioned to accept the insertion of a head of a press stud of a patient electrode. Housing 1424 may be formed from any suitable non-conductive material, including polymeric material. The connector 1400 includes an engagement member 1436 having an actuation surface 1439, which may be a contoured pushbutton, and an engaging face 1437. Engagement member 1436 is pivotable about a pivot 1415 to enable the engaging face 1437 to move from a first position whereby engaging face 1437 is closer to a top portion 1425 of opening 1434 and a second position whereby engaging face 1437 is further from a top portion 1425 of opening 1434. By this arrangement, the bulbous head of a press stud that has been introduced into opening 1434 may be captured in opening 1434 between engaging face 1437 and a sidewall of opening 1434. Engagement member 1436 includes a stiffener 1438, that may have an arcuate shape, disposed between engaging face 1437 and pivot 1415.

The interior recessed surface 1431 of housing 1424 includes a radiolucent conductor 1432 that facilitates the conduction of biological signals between a press stud captured within opening 1434 and a lead wire conductor 1477. Radiolucent conductor 1432 may be included with surface 1431 by any suitable manner, including without limitation, as a conductive coating and/or a conductive material incorporated within housing 1424 or associated portions thereof. In some embodiments, radiolucent conductor 1432 may be formed by dispersing conductive carbon powder over interior recessed surface 1431. The conductive carbon powder is then fused via the application of heat and/or pressure to the polymeric material that forms interior recessed surface 1431. In some embodiments, radiolucent conductor 1432 may be formed by the application of radiolucent conductive ink to interior recessed surface 1431. In other embodiments, the radiolucent conductor 1432 may comprise a carbon fiber wire fixed to the recessed surface 1431. As shown in FIG. 1C, radiolucent conductor 1432 may extend onto at least a portion of a sidewall 1441 of opening 1434.

ECG electrode connector 1400 includes a lead wire 1475 extending from a proximal (e.g., bottom) end thereof. Lead wire 1475 includes an outer insulator 1476 coaxially disposed about a conductor 1477. Conductor 1477 is formed from radiolucent electrically conductive material, such as conductive carbon or conductive carbon monofilament wire. In some embodiments, conductor 1477 is formed from one or more carbon fibers. A distal portion of the outer insulator is stripped thus exposing a distal portion of conductor 1477′. The exposed portion 1477′ of conductor 1477 is operatively joined to radiolucent conductor 1432 of interior recessed surface 1431. Conductor 1477′ may be joined by any suitable manner, including without limitation a crimping element 1478 and/or by radiolucent electrically conductive adhesive. In some embodiments, the exposed portion 1477′ of conductor 1477 and radiolucent conductor 1432 are integrally formed. A strain relief 1479 surrounds a portion of lead wire 1475 where lead wire 1475 exits the housing 1424.

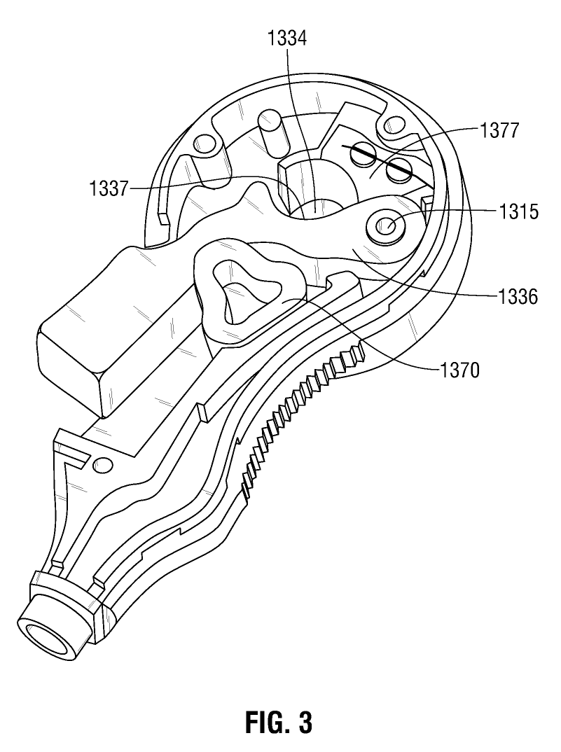

A resilient member 1470 biases engagement member 1436 towards a first position whereby engaging face 1437 is closer to a top portion 1425 of opening 1434. Lobed resilient member 1470 is positioned between a recess 1428 defined in engagement member 1436 and a saddle 1472 provided by housing 1424. Resilient member 1470 may be formed from a radiolucent elastomer, including without limitation, silicone. Resilient member 1470 may have any shape to provide sufficient force to allow the desired movement of the engagement member 1436. The resilient member 1470 may have any regular or irregular shape, including circle, square, triangle, and clover. In one, one embodiment, resilient member 1470 is a lobed member. In the embodiment shown in FIGS. 3A and 3B, lobed resilient member 1470 includes a three-lobe profile having each lobe evenly spaced at about 120° apart, however, a lobed resilient member 1470 in accordance with the present disclosure may include fewer than three lobes, or more than three lobes. Additionally or alternatively, lobed resilient member 1470 may include lobes that are not evenly spaced and/or irregularly placed. The resilient member may be solid throughout, or comprise one or more openings. Lobed resilient member 1470 includes a center opening 1471 defined therein and having a shape that generally corresponds to the contour of the perimeter (e.g., the lobe profile) of lobed resilient member 1470, and/or that may include one or more interior projections 1481. The ratio of the size of opening 1471 to the overall size of the lobed resilient member 1470 determines, at least in part, the resiliency of lobed resilient member 1470 and may facilitate tactile feedback to a user during the actuation/compression and release/extension of the combination of lobed resilient member 1470 and engagement member 1436. For example, and without limitation, cooperative interference between one or more interior projections 1481 as resilient member 1470 is compressed and/or released may generate one or more vibrations that may, in turn, be sensed as tactile feedback by a user's fingertip via actuating surface 1439 and/or via housing 1424.

During use, a user may apply force to actuating surface 1439 using, e.g., a fingertip, thereby overcoming the biasing force of resilient member 1470 to cause engagement member 1436 to rotate slightly counterclockwise about pivot 1415. In turn, engaging face 1437 moves further from a top surface 1425 of opening 1434 which provides sufficient clearance to enable the introduction of a bulbous head of a press stud into opening 1434. Once the press stud is inserted into opening 1434, the user may remove finger pressure from actuating surface 1439, whereupon the biasing force of resilient member 1470 causes engagement member 1436 to rotate slightly clockwise about pivot 1415, thereby electromechanically engaging the press stud with a portion of opening 1434 and thus, electrically coupling the press stud with radiolucent conductor 1432 and conductor 1477.

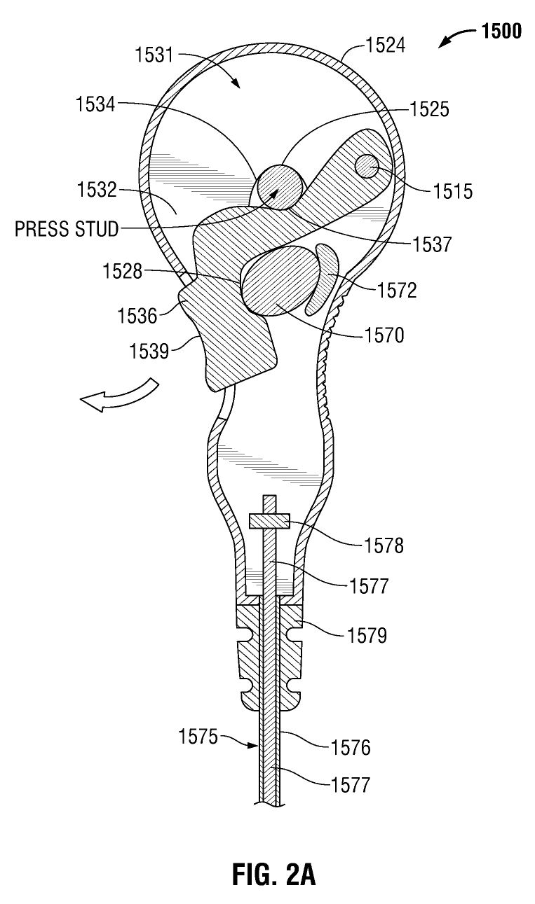

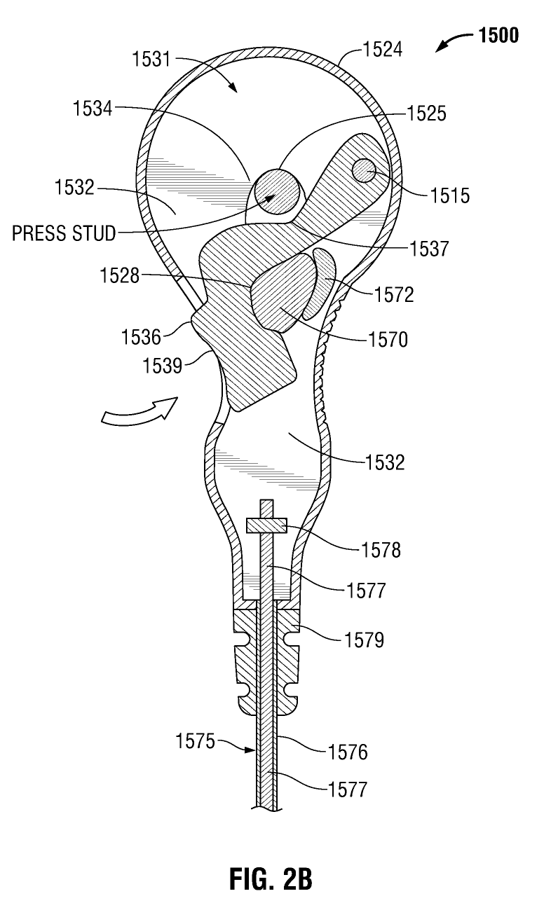

Yet another embodiment of a radiolucent ECG electrode connector 1500 is shown in FIGS. 2A and 2B. In view thereof, and so as not to obscure the present disclosure with redundant information, only those features distinct to ECG electrode connector 1500 will be described hereinafter. Radiolucent electrode connector 1500 includes an engagement member 1536 having an actuation surface 1539, which may be a contoured pushbutton, and an engaging face 1537. Engagement member 1536 is pivotable about a pivot 1515 to enable the engaging face 1537 to move from a first position whereby engaging face 1537 is closer to a top portion 1525 of opening 1534 and a second position whereby engaging face 1537 is further from a top portion 1525 of opening 1534. By this arrangement, the bulbous head of a press stud that has been introduced into opening 1534 may be captured between engaging face 1537 and opening 1534.

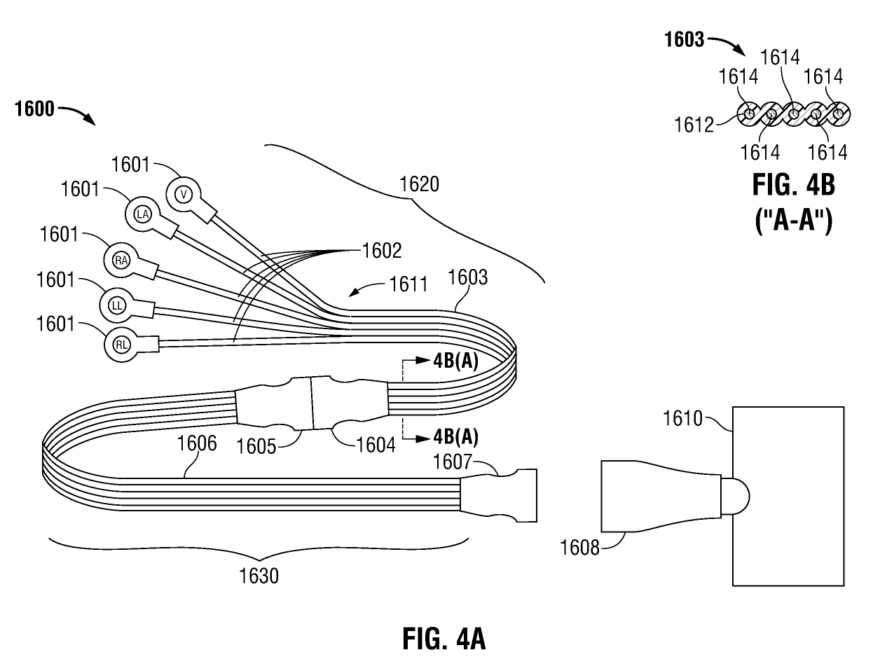

A resilient member 1570 biases engagement member 1536 towards a first position whereby engaging face 1537 is closer to a top portion 1525 of opening 1534. Resilient member 1570 may have any shape to provide sufficient force to allow the desired movement of the engagement member 1536. The resilient member 1570 may have any regular or irregular shape, including circle, square, triangle, and clover, and may, but need not be solid throughout. In some embodiments resilient member 1570 has a generally spherical shape. Spherical resilient member 1570 is positioned between a recess 1528 defined in engagement member 1536 and a saddle 1572 provided by a housing 1524. Spherical resilient member 1570 may be formed from a radiolucent elastomer, including without limitation, silicone. In the embodiment shown in FIGS. 4A and 4B, spherical resilient member 1470 may include surface or internal features, such as without limitation, ribs, voids, and/or textures that may facilitate tactile feedback to a user during the actuation/compression and release/extension of the combination of spherical resilient member 1570 and engagement member 1536. In some embodiments resilient member 1570 may have a generally cylindrical shape, a generally ovoid shape, and/or or a compound shape that may include, e.g., a combination spherical, cylindrical, and/or ovoid shape. In some embodiments, resilient member 1570 may be hollow.

FIG. 3 shows in another embodiment of the present invention similar to the electrode connector shown in FIGS. 1A, 1B, and 1C. In view thereof, and so as not to obscure the present disclosure with redundant information, only those features distinct to ECG electrode connector 1300 will be described hereinafter. As seen in FIG. 3, opening 1334 which is dimensioned to accept the insertion of a head of a press stud of a patient electrode is bounded on at least one side by a conductor 1377. Conductor 1377 may have any size and shape as long as at least a portion of the conductor extend into opening 1334 along at least a portion of sidewall 1334. In one embodiment, conductor 1377 extends through opening 1334 to completely cover at least apportion of the circumference of the opening 1334. Conductor 1377 may be made of a radiolucent conductive material such as a conductive polymer or a conductive carbon. A radiolucent leadwire (not shown) formed of a conductive carbon may be positioned in a passageway 1399 of the connector housing and joined to conductor 1377. In use, once an electrode stud is positioned in opening 1334 and engagement member 1336 is released, engagement face 1337 captures the electrode stud between the engagement face 1337 and a portion of conductor 1377.

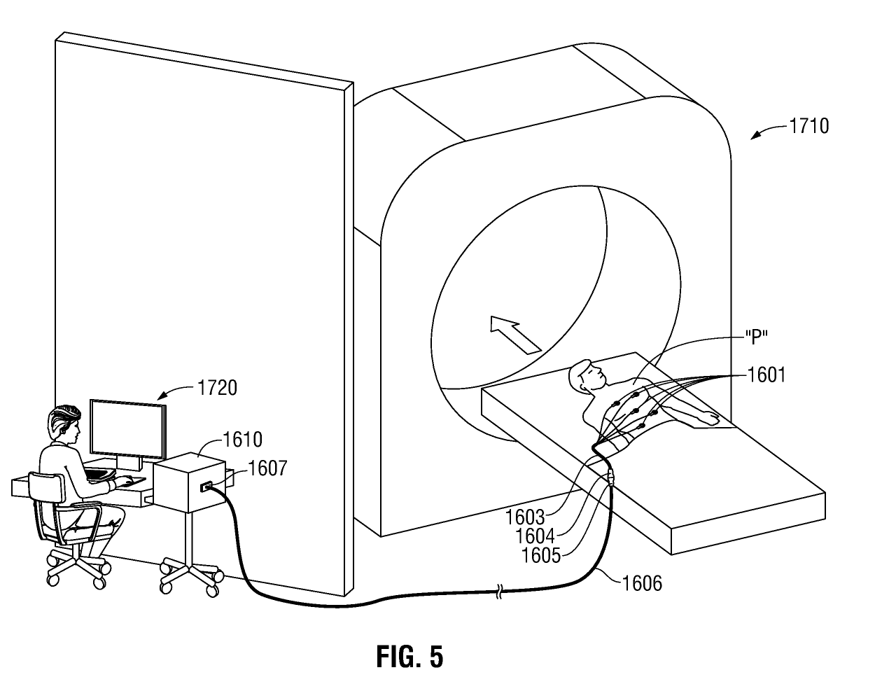

Turning now to FIGS. 4A, 4B, and 5, another aspect of the present disclosure is illustrated wherein a radiolucent ECG lead system 1600 for use with an imaging system 1610 is provided. The radiolucent ECG lead system 1600 includes a radiolucent ECG lead set assembly 1620. Radiolucent ECG lead set assembly 1620 includes one or more radiolucent ECG lead set cables 1602 having a length, and one or more radiolucent electrode connectors 1601 operatively joined to a distal end of an ECG lead set cable 1603. The ECG lead set cables 1603 includes a plurality of individual radiolucent wires 1602 , such as conductive carbon wires, arranged in a ribbon-cable configuration as shown in FIGS. 4A and 14B. The individual radiolucent wires 1602 separate from the ribbon 1603 at a separation point 1611 positioned between a distal end and a proximal end of radiolucent ECG lead set assembly 1620. It is understood that the separation point may vary and may be determined at the point of use, wherein the user separates the ribbon to a desired length for a particular application. In some embodiments, separation point 1611 is positioned about halfway between a distal end and a proximal end of radiolucent ECG lead set assembly 1620. In some embodiments, the one or more radiolucent electrode connectors include radiolucent ECG electrode connector 1300, radiolucent ECG electrode connector 1400, and/or radiolucent ECG electrode connector 1500. The one or more electrode connectors 1601 are configured to electrically connect to electrodes placed on a patient, and to an intermediate lead set connector 1604 disposed at a proximal end of the ECG lead set cable 1620.

The radiolucent ECG lead set cables 1602 include a center conductor 1614 and an outer insulator 1612. Center conductor 1614 is formed from a radiolucent electrically conductive material, including without limitation one or more carbon fibers. The one or more carbon fibers may be combined with other materials, including without limitation, polypropylene, polycarbonate, polyethylene, polyurethane, or polytetrafluoroethylene fibers to increase strength and/or flexibility of the conductor and the overall cable assembly 1620.

The ECG lead system 1600 further includes an ECG lead extension assembly 1630. ECG lead extension assembly 1630 includes an ECG lead extension cable 1606, which may be configured as a ribbon cable as shown in FIG. 4A, and/or may be configured in any other suitable cable arrangement. Lead extension cable 1606 may but need not be formed of radiolucent materials. In one embodiment, lead extension cable 1606 is comprises wires formed of conventional tinned copper since it is outside of the imaging area. Limiting the use of radiolucent wires to areas within and adjacent the imaging area and connecting the radiolucent lead wires to a conventional lead extension cable may reduce the cost associated with providing longer radiolucent cables. Reducing the length of radiolucent lead wires may also increase durability since conventional tinned copper wires may be stronger than conductive carbon wires. In some embodiments, ECG lead extension cable 1606 may have a length greater that the length of the ECG lead set cable 1620. An ECG lead set extension connector 1605 is disposed at a distal end of the ECG lead extension cable 1630. ECG lead set extension connector 1605 is configured and adapted to mate with and electrically connect to the intermediate lead set connector 1604 that is disposed at a proximal end of the ECG lead set cable 1620. A device connector 1607 disposed at a proximal end of the ECG lead extension cable 1630. Device connector 1607 is configured and adapted to mate with and electrically connect to an ECG monitor 1610. Additionally or alternatively, an adapter 1608 may be configured and adapted to mate with, and operably couple to, device connector 1607. Adapter 1608 is configured to enable operable coupling or interfacing between device connector 1607 and an ECG monitor 1610 that would otherwise be incompatible with the electrical or physical configuration of device connector 1607.

In use, a patient P undergoing an imaging procedure by an imaging apparatus 1710 may be connected to an ECG monitor 1610 by ECG lead set assembly 1620. The ECG electrode connectors 1601 are coupled to press stud pads (not explicitly shown) attached to the patient P. ECG lead set assembly 1620 is coupled via intermediate lead set connector 1604 and extension connector 1605 to ECG lead extension assembly 1630. ECG lead extension assembly 1630, in turn, is coupled to the ECG monitor 1610, which may be positioned in a control suite adjacent to imaging station 1720.

It will be understood that various modifications may be made to the embodiments disclosed herein. Further variations of the above-disclosed and other features and functions, or alternatives thereof, may be desirably combined into many other different systems, instruments and applications. Various presently unforeseen or unanticipated alternatives, modifications, variations or improvements therein may be subsequently made by those skilled in the art, which are also intended to be encompassed by the following claims.

What is claimed is:

1. An ECG lead system, comprising:
a radiolucent ECG lead set assembly, comprising:
a radiolucent ECG lead set cable comprising at least one radiolucent conductor;
at least one radiolucent electrode connector operatively coupled to a distal end of the ECG lead set cable; and
an ECG intermediate lead set connector disposed at a proximal end of the ECG lead set cable; and
an ECG lead extension assembly, comprising:
an ECG lead extension cable comprising at least one conductor;
an ECG lead set extension connector disposed at a distal end of the ECG lead extension cable; and
a device connector disposed at a proximal end of the ECG lead extension cable;
wherein the ECG intermediate lead set connector is configured to operatively couple to the ECG lead set extension connector.

2. The ECG lead system in accordance with claim 1, wherein the at least one radiolucent conductor of the radiolucent ECG lead set cable is formed from carbon fiber.

3. The ECG lead system in accordance with claim 1, wherein the at least one radiolucent conductor is arranged in a ribbon-cable configuration.

4. The ECG lead system in accordance with claim 3, wherein the at least one radiolucent conductor separates from the ribbon-cable configuration at a separation point.

5. The ECG lead system in accordance with claim 4, wherein the separation point is positioned about halfway between a distal end and a proximal end of radiolucent ECG lead set assembly.

6. The ECG lead system in accordance with claim 1, wherein the device connector is configured to operably couple to an ECG monitor.

7. The ECG lead system in accordance with claim 1, further comprising an adapter configured to enable operable coupling of the device connector to an ECG monitor that is incompatible with the device connector.

8. A method of performing an ECG, comprising:
providing one or more radiolucent ECG connectors, comprising:
a housing having an interior recessed surface having disposed therein an opening dimensioned to operably receive the press stud of an ECG electrode pad;
a radiolucent conductor disposed on at least a portion of the interior recessed surface;
a radiolucent lead wire conductor extending from a proximal end of the housing and operably coupled to the radiolucent conductor; and
an engagement member pivotably disposed upon the interior recessed surface and having an engaging face and a pivot, wherein the engagement member is pivotable between a first position whereby the engaging face is closer to a top portion of the opening and a second position whereby engaging face is further from a top portion of the opening;
providing a radiolucent ECG lead system, comprising:
a radiolucent ECG lead set assembly, comprising:
a radiolucent ECG lead set cable comprising at least one radiolucent conductor;
at least one radiolucent electrode connector operatively coupled to a distal end of the ECG lead set cable; and
an ECG intermediate lead set connector disposed at a proximal end of the ECG lead set cable; and
an ECG lead extension assembly, comprising:
an ECG lead extension cable comprising at least one conductor;
an ECG lead set extension connector disposed at a distal end of the ECG lead extension cable; and
a device connector disposed at a proximal end of the ECG lead extension cable;
wherein the ECG intermediate lead set connector is configured to operatively couple to the ECG lead set extension connector.
attaching one or more electrode pads to the body of a patient;
operatively coupling the one or more radiolucent ECG connectors to a corresponding one of the one or more electrode pads;
operatively coupling the device connector to an ECG monitor;
imaging the patient in an imaging apparatus selected from the group consisting of an MRI scanner, a CT scanner, and a PET scanner; and
monitoring an ECG of the patient on the ECG monitor.

9. The method in accordance with claim 8, further comprising coupling the ECG intermediate lead set connector to the ECG lead set extension connector.

10. The method in accordance with claim 8, further comprising:
providing an adapter configured to enable operable coupling of the device connector to an ECG monitor;
coupling the device connector to the adapter; and
coupling the adapter to the ECG monitor.
